(19) United States
(12) Patent Application Publication
(10) Pub. No.: US 2010/0041933 A1
(43) Pub. Date: Feb. 18, 2010

(54) Catalyst and Process for Hydrocarbon Conversions

(75) Inventors: James R. Butler, League City, TX (US); Xin Xiao, Augusta, GA (US); Rosa Hall, Houston, TX (US); Sivadinarayana Chinta, Missouri City, TX (US)

Correspondence Address: FINA TECHNOLOGY INC, PO BOX 674412, HOUSTON, TX, 77267-4412, US

(73) Assignee: Fina Technology, Inc., Houston, TX (US)

(21) Appl. No.: 12/193,682

(22) Filed: Aug. 18, 2008

(51) Int. Cl. C07C 5/52; B01J 29/072; B01J 29/068; B01J 21/12

(52) U.S. Cl. 585/475; 502/74; 502/66

(57) ABSTRACT

A nickel-mordenite catalyst promoted with Rhodium that is useful in the conversion of hydrocarbons is disclosed. The catalyst and methods for its use can provide hydrocarbon conversion with an extended catalyst life as compared to nickel-mordenite catalyst not promoted with Rhodium.

### CROSS-REFERENCE TO RELATED APPLICATIONS

Not applicable.

### FIELD

This invention relates generally to catalysts and processes for hydrocarbon conversions and more particularly to the disproportionation of alkylaromatic feedstreams.

### BACKGROUND

The disproportionation of toluene involves a well known transalkylation reaction in which toluene is converted to benzene and xylene, often referred to as a Toluene Disproportionation Process or TDP, in accordance with the following reaction:

Toluene Disproportionation: Toluene⇄Benzene+Xylene (1)

Mordenite is one of a number of molecular sieve catalysts useful in the transalkylation of alkylaromatic compounds. Mordenite is a crystalline aluminosilicate zeolite exhibiting a network of silicon and aluminum atoms interlinked by oxygen atoms within the crystalline structure. For a general description of mordenite catalysts, reference is made to Kirk-Othmer, Encyclopedia of Chemical Technology, 3rd Edition, 1981, under the heading “Molecular Sieves”, Vol. 15, pages 638-643, incorporated by reference herein. Mordenite, as found in nature or as synthesized to replicate the naturally occurring zeolite, typically exhibits a relatively low silica-to-alumina mole ratio of about 10 or less. Also known, however, are mordenite catalysts exhibiting substantially lower alumina content. These alumina deficient mordenite catalysts exhibit silica-to-alumina ratios greater than 10, ranging up to about 100, and may be prepared by direct synthesis as disclosed, for example, in U.S. Pat. No. 3,436,174 to Sand or by acid extraction of a more conventionally prepared mordenite as disclosed in U.S. Pat. No. 3,480,539 to Voorhies et al, both of which are incorporated by reference herein. Both the typical and the aluminum deficient mordenites are known to be useful in the disproportionation of toluene.

Disproportionation of toluene feedstock may be performed at temperatures ranging from about 200° C. to about 600° C. or above and at pressures ranging from atmospheric to perhaps 100 atmospheres or above and at liquid hourly space velocities (LHSV) typically in the range of around 0.1 hr⁻¹ to 10 hr⁻¹. The specific catalyst, however, may impose constraints on reaction temperatures in terms of catalyst activity and aging characteristics. In general relatively high temperatures are used when employing the high aluminum mordenites (low silica-to-alumina ratios) and somewhat lower temperatures when employing the low alumina mordenites. Accordingly, where mordenite catalysts exhibiting high silica/alumina ratios have been employed in the transalkylation of alkylaromatics, it has been the practice to operate toward the lower end of the temperature range.

Hydrogen is generally supplied along with toluene to the reaction zone. While the disproportionation reaction (1) does not involve chemical consumption of hydrogen, the use of a hydrogen co-feed is generally considered to prolong the useful life of the catalyst. The amount of hydrogen supplied, which normally is measured in terms of the hydrogen/toluene mole ratio, is generally shown in the prior art to increase as temperature increases. The hydrogen:toluene mole ratio can generally range from 0.05:1 to 5:1.

Another method of producing benzene and xylene is by processing heavier aromatic compounds, i.e. aromatic compounds of C₈ or greater, that have lesser value than benzene and xylene, such as those produced from hydrocarbon reforming processes.

Conventional TDP processes utilizing Ni-Mordenite catalysts may exhibit Ni agglomeration, otherwise referred to as sintering, over the catalyst life. This agglomeration of the nickel reduces the distribution of the nickel throughout the catalyst, thereby reducing the beneficial results of having nickel distribution within the catalyst, resulting in reduced catalyst activity. This may also result in reducing the effective catalyst life, the need for more frequent regeneration, and an inability to effectively regenerate the catalyst.

In view of the above, it would be desirable to have a process of conducting toluene disproportionation and/or conversion of heavy aromatic compounds with a nickel-mordenite catalyst without the significant adverse effect on catalyst activity or catalyst life that comes from Ni sintering.

### SUMMARY

One embodiment of the present invention is a catalyst useful in the conversion of hydrocarbons that includes a molecular sieve catalyst promoted with at least 0.005% by weight rhodium. The molecular sieve catalyst can be a mordenite zeolite having at least 0.5% by weight nickel. The nickel content can be between 0.5 wt % and 1.5 wt % and the rhodium content can be between 0.005 wt % and 1.5 wt %. The catalyst can have a silica to alumina molar ratio of from about 10:1 to about 100:1.

The catalyst can be used in a process for the disproportionation of toluene to benzene and xylene that includes passing a toluene/hydrogen feedstock over the catalyst at reaction conditions sufficient to provide toluene conversion at a rate of about at least 30 percent and the catalyst exhibits extended catalyst life over nickel-mordenite catalyst not promoted with rhodium. The hydrogen:toluene molar ratio can range between 0.05:1 to 5:1. The benzene:xylene ratio by weight in the product stream can be greater than 0.85

The reaction temperature can range from 150° C. to 500° C. and the reaction temperature can be adjusted to maintain a toluene conversion level of at least 40 percent. The reaction pressure can range between 200 psig to 800 psig.

The toluene conversion reaction can continue with a toluene conversion of at least 30 percent for at least 20 days with no more than 15° C. reactor temperature increase. In an alternate embodiment the toluene conversion reaction can continue with a toluene conversion of at least 40 percent for at least 20 days with no more than 10° C. reaction temperature increase.

The catalyst can have an extended catalyst life by a factor of at least two times over nickel-mordenite catalyst not promoted with rhodium. The average catalyst deactivation can be 0.5° C. per day or less.

In an alternate embodiment the catalyst can also be used in a process for the conversion of a feed of heavy aromatics composed primarily of C₈₊ alkylaromatic compounds to produce products of benzene, toluene and xylene. The process includes providing a reaction zone containing the nickel-mordenite catalyst promoted with rhodium and introducing a feed comprising heavy aromatics composed primarily of C₈₊ alkylaromatic compounds at reaction zone conditions and removing conversion products from the reaction zone and the catalyst exhibits extended catalyst life over nickel-mordenite catalyst not promoted with rhodium.

Toluene feed can also be introduced into the reaction zone along with the heavy aromatic feed. In some embodiments the heavy aromatics make up substantially the entire feed introduced into the reaction zone, while in others the heavy aromatics make up at least 75% by total weight of the feed introduced into the reaction zone.

The reaction zone can be operated at a temperature of from about 250° C. to about 500° C., and a pressure of at least 200 psig. In one embodiment the average catalyst deactivation is no more than 0.5° C. per day. In another embodiment the catalyst exhibits extended catalyst life by a factor of at least two times over nickel-mordenite catalyst not promoted with rhodium.

The conversion of a feed of heavy aromatics can further include introducing a first feed comprising substantially pure toluene feedstock into the reaction zone so that the first feed contacts the catalyst under initial reaction zone conditions selected for the disproportionation of substantially pure toluene to obtain a target toluene conversion between 30% and 55%. A second feed comprising heavy aromatics composed primarily of C₈₊ alkylaromatic compounds is introduced, allowing conversion of the second feed while the reaction zone is at the reaction zone conditions selected for the disproportionation of the pure toluene.

### BRIEF DESCRIPTION OF DRAWINGS

FIG. 1 illustrates experimental results of toluene conversion and reaction temperature when a rhodium promoted nickel mordenite catalyst is used in a toluene disproportionation reaction.

FIG. 2 illustrates comparative experimental results of toluene conversion and reaction temperature when a nickel mordenite catalyst without rhodium is used in a toluene disproportionation reaction.

FIG. 3 illustrates additional experimental data of toluene conversion and reaction temperature when a 0.01 wt % rhodium promoted nickel mordenite catalyst is used in a toluene disproportionation reaction.

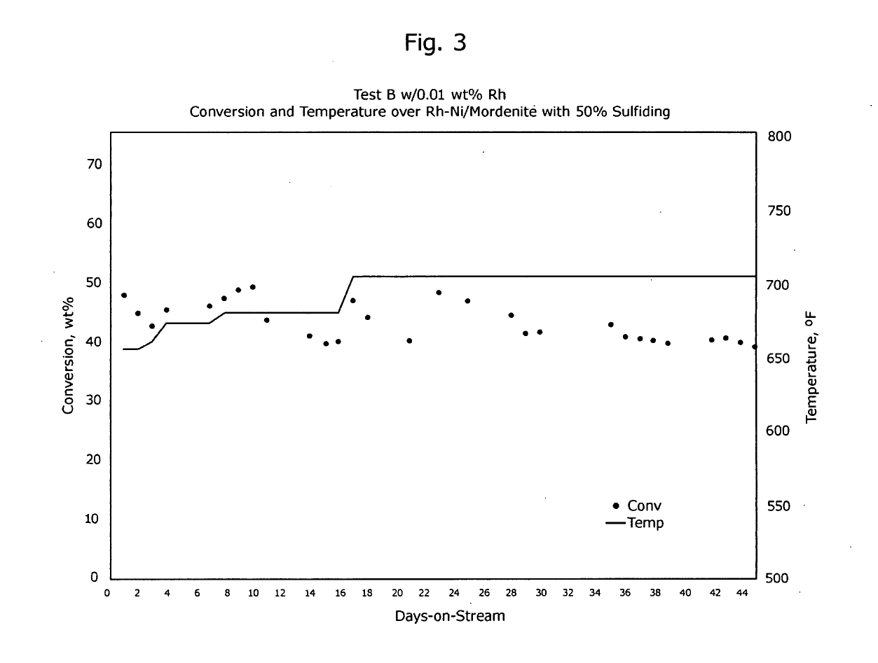

FIG. 4 illustrates additional experimental data of toluene conversion and reaction temperature when a 0.05 wt % rhodium promoted nickel mordenite catalyst is used in a toluene disproportionation reaction.

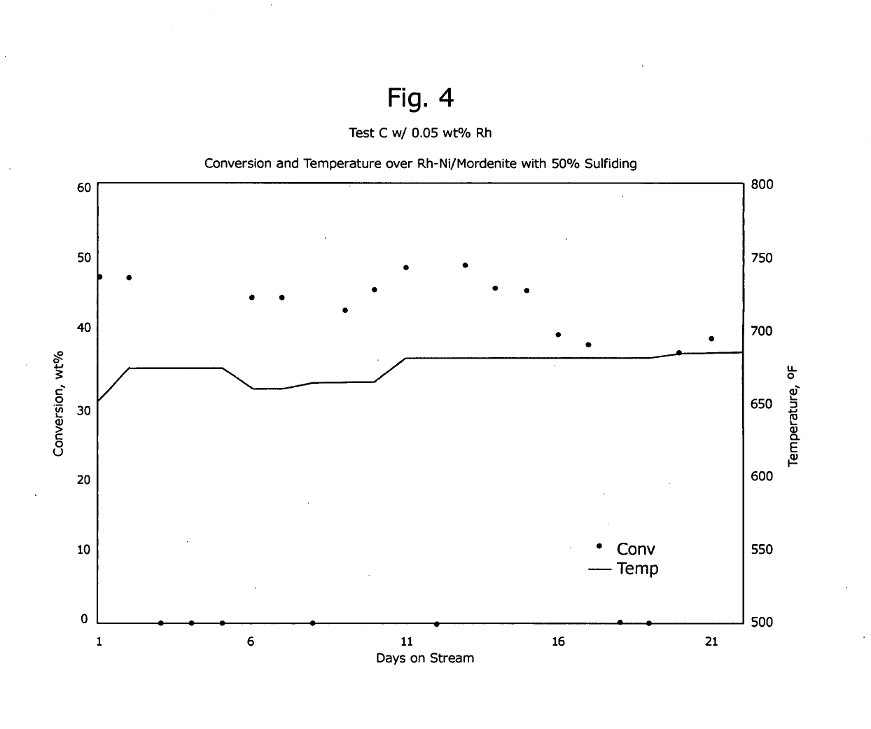

### DETAILED DESCRIPTION

The use of nickel-mordenite molecular sieve catalysts in toluene disproportionation and heavy aromatic conversion reactions is well known in the art. The present invention provides an improved means of conducting these reactions whereby the catalyst deactivation typically found with a metal modified mordenite catalyst, such as a nickel-mordenite catalyst, is reduced.

In accordance with the present invention, there is provided a metal promoted molecular sieve catalyst for the conversion of hydrocarbons in which catalyst activity and aging quality are enhanced. It is well known in the art that mordenite can be modified with the addition of metals such as nickel, palladium or platinum. These catalysts can exhibit reduced catalyst activity, shortened catalyst life and an inability to effectively regenerate the catalyst possibly due to agglomeration or sintering of the metals over the catalyst life. Testing was conducted to examine the effects of the addition of rhodium (Rh) to a standard Ni/Mordenite catalyst on the catalyst life.

Rhodium was added to a Ni/Mordenite catalyst and tested using both toluene and C₉ feeds at TDP conditions. The Rh promoter was found to extend the catalyst life over a Ni/Mordenite based TDP catalyst without the Rh promoter and be successful in the conversion of heavy aromatics.

In one embodiment the rhodium content of the modified Ni/Mordenite catalyst can range from 0.005 wt % to 1.5 wt % of the total catalyst. In alternate embodiments the rhodium content of the modified Ni/Mordenite catalyst can range from 0.01 wt % to 1.0 wt % of the total catalyst; or from 0.01 wt % to 0.08 wt % of the total catalyst; or from 0.02 wt % to 0.05 wt % of the total catalyst. In one embodiment the nickel content of the base NiAMordenite catalyst can range from 0.25 wt % to 2.0 wt % of the total catalyst. In alternate embodiments the nickel content of the base Ni/Mordenite catalyst can range from 0.5 wt % to 1.5 wt % of the total catalyst; or from 0.75 wt % to 1.25 wt % of the total catalyst.

Hydrogen is supplied along with the toluene to the reaction zone, typically at a hydrogen:toluene mole ratio of 4:1 or less. The initial toluene conversion rate is generally set at a level of at least 40% with an initial steady state reactor temperature (as measured at the reactor inlet) within the range of 150° C.-471° C. (300° F.-880° F.), often between 315° C.-385° C. (600° F.-725° F.), and generally having a temperature gradient across the reactor of no more than 27° C. (50° F.). The process is continued at a generally stable toluene conversion rate of at least 40% while retaining the activity of the catalyst, as indicated by toluene conversion, with a progressive incremental temperature increase. It is desirable to have the temperature increase as low as possible to maintain reactor severity, such as less than 0.5° C. rise per day, or less than 5.5° C. (10° F.) per week, or no more than 2.8° C. (5° F.) per week as normalized by changes in space velocity of the toluene feedstock over the catalyst bed. The reaction pressure will generally range between 100 psig to 1200 psig, can range between 200 psig to 800 psig, and can range from 500 psig to 700 psig.

In a further embodiment of the invention, there is provided a toluene disproportionation process that is initiated by establishing a hydrogen environment in a catalytic reaction zone containing a Ni/Mordenite disproportionation catalyst modified by the promotion of rhodium. The hydrogen environment is established at a reaction zone temperature substantially less than an intermediate temperature within the range of about 121° C.-260° C. (250° F.-500° F.). The reaction zone is progressively heated, while maintaining the reaction zone under a hydrogen environment, until the intermediate temperature as described above is reached. Once the intermediate temperature range is reached, hydrogen flow through the reactor is continued for a period of several hours, normally about 4-10 hours. Thereafter, a toluene feedstock is supplied to the reaction zone along with hydrogen, typically to provide a hydrogen:toluene mole ratio within the range of 1:1 to 4:1. After initiating the toluene feed, the reaction zone is further heated from the intermediate temperature to a higher initial toluene disproportionation temperature at which toluene conversion is at least 40%. The hydrogen:toluene mole ratio normally will be maintained relatively constant as the temperature is increased. The initial disproportionation temperature should be less than 426° C. (800° F.) and more typically within the range of 315° C.-371° C. (600° F.-700° F.). Typically, the reaction zone temperature, when the hydrogen environment is initiated, is no more than 65° C. (150° F.) and the reaction zone temperature is increased from the initial temperature to the intermediate temperature over a time period of at least 2 hours. Typically, the initial reaction zone temperature will be at ambient temperature.

### EXAMPLE

A Ni/Mordenite disproportionation catalyst was modified with the addition of 420 ppm Rh (0.042 wt %) and loaded into a catalytic reaction zone. At the conclusion of the initial transient conditions accompanying the initiation of toluene feed to the reaction zone, initial steady state conditions for disproportionation of toluene to benzene and xylene were established. The reactor was operated to maintain a generally consistent reactor severity and toluene conversion. The inlet reactor pressure was approximately 600 psig. The reactor temperature was found to hold steady, being 354° C. (670° F.) on day 2 as it was on day 23 when both conversions were 47%, thereby not indicating catalyst deactivation as would normally be expected. The temperature of the Ni/Mordenite base catalyst without the Rh promoter under similar conditions would show an increase in temperature during the same time period, indicating catalyst deactivation.

In one experiment a Ni/Mordenite catalyst with 1 wt % nickel, Zeolyst CP-751 from Zeolyst International of Valley Forge, Pa., USA, was used as the base material. Rhodium was added using an incipient wetness method with an aqueous solution of RhCl₃.H₂O salt, dried at 110° C., and then calcined at 550° C. for 2 hr. The catalyst was measured to have 420 ppm Rh impregnation.

The TDP performance was evaluated in a lab scale reactor. The testing conditions are summarized as following.

| | |
|---|---|
| Reactor, down flow | Rh promoted Ni/Mordenite catalyst |
| Feed | Toluene |
| LHSV | 3/hr |
| H2/HC molar ratio | 1:1 then 3:1 |
| Temperature | Adjusted to hold constant conversion |
| RX Inlet Pressure | 600 psig |
| Target conversion | 47 ± 1% (53% toluene in effluent) |
| Catalyst volume | 30 ml, 14-20 mesh without dilution |

Initially the startup used was 1:1 H₂/oil molar ratio without sulfiding. The system pressure decreased due to very high hydrogen consumption. The hydrogen rate was increased to 3:1 H₂/oil ratio at about 280° C. bed temperature during the temperature ramp from 250° C. to 350° C. at 6° C./hr. The effluent sample was analyzed at 10% nonaromatics. The catalyst was then sulfided the next day using DMDS to have 50 mol % sulfur relative to the catalyst nickel.

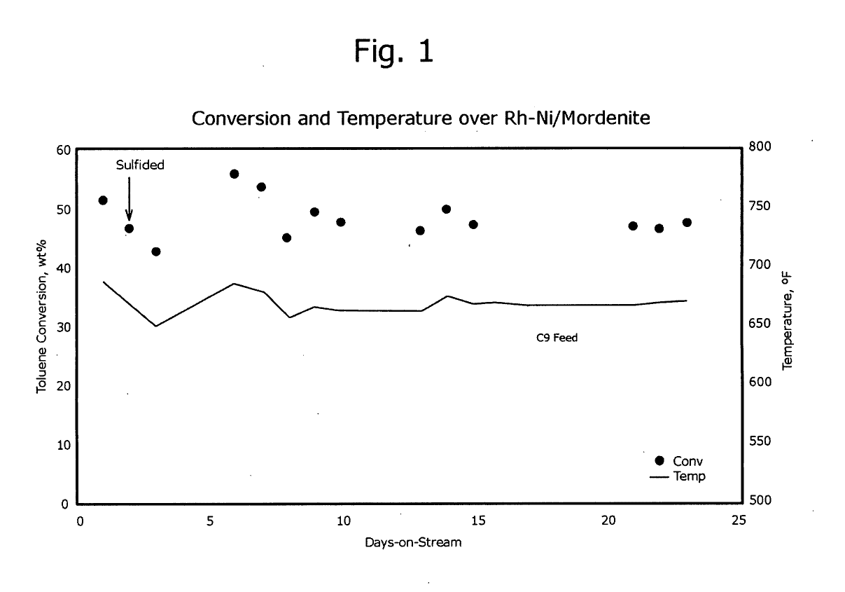

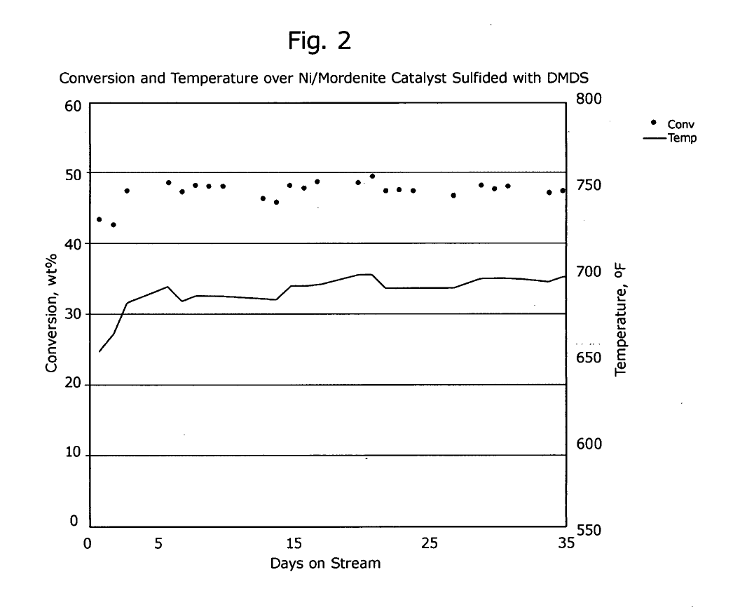

FIG. 1 shows the toluene conversion and bed temperature during the study. The bed temperature was the same at 354° C. (670° F.) on day 2 and day 23 when both conversions were 47%, while the temperature of the Ni/Mordenite base without Rh addition would increase by about 0.5° C. per day at comparable conditions as can be seen in FIG. 2 and from the data in Table 4.

A C₉ aromatic mixture was used as feed replacing toluene between days 16 and 20. The toluene feedstream was then used for the remainder of the experiment with results consistent with those obtained prior to the C₉ aromatic feed. The feed and effluent compositions are averaged for each feed in Table 1. There were 4% to 6% nonaromatics in the liquid effluent stream using either toluene or C₉ aromatic feed. The high activity and stability indicated the in-house impregnation was efficient to have a dispersed metal loading.

The C₉ aromatic mixture feed had only 9.7% of benzene/toluene/xylene aromatics (BTX) content (thought to be mostly o-xylene). The effluent from the reaction had a total of 40.9% BTX, therefore BTX aromatics were generated across the catalyst bed with the C₉ aromatic feed. The TMB (trimethylbenzene) and ET (ethyltoluene) conversions were 34.4 and 49.8%, respectively. The off-gas hydrocarbon has 50.3% propane, 38.6% ethane, 6.3% butane, and 3.7% methane.

In the following tables all values are in wt % unless designated otherwise.

TABLE 1
Feed and Liquid Effluent Composition over 420 ppm Rh—Ni/Mordenite
Catalyst

| | TDP | | GRU OH | | TDP | |
|---|---|---|---|---|---|---|
| Component | Feed | Effluent | Feed | Effluent | Feed | Effluent |
| n-Ar | 0.08 | 4.97 | 0.05 | 6.08 | 0.07 | 5.75 |
| Benzene | 0.01 | 16.58 | 0.00 | 2.26 | 0.01 | 15.05 |
| Toluene | 99.91 | 51.25 | 0.47 | 11.92 | 99.71 | 54.09 |
| EB | 0.00 | 0.76 | 0.10 | 3.58 | 0.00 | 0.74 |
| p-Xylene | 0.00 | 5.00 | 0.60 | 5.60 | 0.00 | 4.65 |
| m-Xylene | 0.00 | 11.00 | 1.57 | 12.34 | 0.00 | 10.22 |
| o-Xylene | 0.00 | 4.58 | 6.96 | 5.22 | 0.11 | 4.25 |
| Cumene | 0.00 | 0.01 | 1.16 | 0.00 | 0.00 | 0.00 |
| n-Pr-BZ | 0.00 | 0.05 | 4.04 | 0.16 | 0.00 | 0.00 |
| ET | 0.00 | 1.50 | 26.42 | 13.83 | 0.09 | 1.57 |
| TMB | 0.00 | 2.94 | 35.12 | 24.05 | 0.01 | 2.55 |
| DEB | 0.00 | 0.00 | 9.88 | 2.74 | 0.00 | 0.00 |
| BuBenzene | 0.00 | 0.00 | 0.00 | 0.00 | 0.00 | 0.00 |
| Other C10 | 0.00 | 0.95 | 13.63 | 12.22 | 0.00 | 0.65 |
| Unidentified | 0.00 | 5.07 | 0.01 | 12.07 | 0.00 | 15.07 |
| Conversions, wt % | | | | | | |
| (Tol + TMB + Et) | | 44.67 | | 23.00 | | 42.70 |
| TMB | | | | 34.36 | | |
| ET | | | | 49.81 | | |
| Toluene | | 48.60 | | | | 46.57 |

The Rh—Ni/Mordenite and NiAMordenite catalysts are compared in Table 2 when processing C₉ aromatic feed. The product yields were relatively similar due to reaction equilibrium. The Rh—Ni/Mordenite showing higher C₁₀ and less C₈ in the effluent was due to higher C₁₀ content (23.5%) in the testing feed.

TABLE 2
C₉ Feed over Ni/Mordenite W/O Rh-Promotor

| | |
|---|---|
| Rh—Ni/Mordenite | Ni/Mordenite |

| Feed | Effluent | | Feed | Effluent |
|---|---|---|---|---|
| 0.05 | 6.08 | n-Ar | 0.02 | 5.52 |
| 0.00 | 2.26 | Benzene | 0.01 | 2.19 |
| 0.47 | 11.92 | Toluene | 1.01 | 12.22 |
| 9.24 | 26.74 | C8 | 12.18 | 38.40 |
| 66.74 | 38.04 | C9 | 76.13 | 40.37 |
| 23.51 | 14.95 | C10 | 6.68 | 4.38 |
| 0.01 | 12.07 | Others | 4.99 | 11.33 |

The Ni/Mordenite catalyst promoted with 420 ppm Rh showed stability in TDP and C₉₊ aromatic conversion applications. The product yields were very nearly the same as the Ni-mordenite catalyst when using a heavy aromatic feed and appears to be an effective catalyst for the conversion of heavy aromatics to BTX. The high activity and stability indicated that the in-house impregnation was very efficient to have a dispersed metal loading.

The following table gives experimental data from the Experiment as shown in FIG. 1.

TABLE 3
Toluene conversion and reactor temperature for Test A 0.01 wt %
Rh—Ni/Mordenite catalyst.

| | Toluene | Temp | Pressure | LHSV | H2/toluene |
|---|---|---|---|---|---|
| Day | conversion wt % | ° F. | psig | Hr⁻¹ | molar |
| 1 | 51.5 | 689 | 608 | 3.1 | 2.9 |
| 2 | 46.9 | 670 | 608 | 2.8 | 3.2 |
| 3 | 42.9 | 652 | 608 | 2.8 | 3.2 |
| 6 | 55.7 | 688 | 608 | 2.8 | 3.2 |
| 7 | 53.7 | 680 | 608 | 2.8 | 3.2 |
| 8 | 44.9 | 658 | 608 | 3.0 | 3.0 |
| 9 | 49.2 | 667 | 608 | 2.8 | 3.2 |
| 10 | 47.5 | 664 | 608 | 2.8 | 3.2 |
| 13 | 45.9 | 663 | 608 | 2.8 | 3.2 |
| 14 | 49.6 | 675 | 608 | 2.8 | 3.2 |
| 15 | 46.9 | 669 | 607 | 2.8 | 3.2 |
| 16 | — | 669 | 608 | 2.8 | 3.2 |
| 17 | — | 667 | 608 | 2.8 | 3.2 |
| 20 | — | 667 | 608 | 2.9 | 3.4 |
| 21 | 46.5 | 666 | 608 | 2.8 | 3.2 |
| 22 | 46.2 | 669 | 608 | 2.8 | 3.6 |
| 23 | 47.0 | 670 | 608 | 2.8 | 3.3 |

Comparative data for disproportionation of toluene to benzene and xylene using a commercial Ni/Mordenite catalyst, Zeolyst CP 751 having no Rhodium is shown below.

TABLE 4
TDP Data using Ni/Mordenite (w/ sulfiding), no Rh

| | Toluene | Temp | Pressure | LHSV | H2/toluene |
|---|---|---|---|---|---|
| Day | conversion wt % | ° F. | psig | Hr⁻¹ | molar |
| 1 | 43.5 | 653 | 590 | 3.0 | 1.2 |
| 2 | 42.7 | 663 | 592 | 3.0 | 1.0 |
| 3 | 47.4 | 682 | 594 | 2.9 | 1.0 |
| 6 | 48.5 | 692 | 592 | 2.9 | 1.0 |
| 7 | 47.3 | 683 | 590 | 2.9 | 3.1 |
| 8 | 48.1 | 686 | 591 | 2.9 | 3.1 |
| 9 | 48.1 | 686 | 591 | 2.9 | 3.1 |
| 10 | 48.1 | 686 | 591 | 2.9 | 3.1 |
| 13 | 46.5 | 684 | 591 | 2.9 | 3.1 |
| 14 | 45.9 | 684 | 591 | 2.9 | 3.1 |
| 15 | 48.2 | 692 | 590 | 2.9 | 3.1 |
| 16 | 47.8 | 692 | 591 | 2.9 | 3.1 |
| 17 | 48.7 | 693 | 591 | 3.0 | 3.0 |
| 20 | 48.6 | 698 | 592 | 2.9 | 3.1 |
| 21 | 49.3 | 698 | 592 | 2.9 | 3.1 |
| 22 | 47.4 | 690 | 592 | 3.0 | 3.0 |
| 23 | 47.4 | 691 | 592 | 2.9 | 3.1 |
| 24 | 47.3 | 690 | 592 | 3.0 | 3.0 |
| 27 | 46.7 | 690 | 592 | 3.0 | 3.0 |
| 29 | 48.1 | 696 | 591 | 3.0 | 3.0 |
| 30 | 47.7 | 696 | 592 | 3.0 | 3.0 |
| 31 | 48.1 | 696 | 592 | 3.0 | 3.0 |
| 34 | 47.0 | 694 | 592 | 3.0 | 3.0 |
| 35 | 47.3 | 697 | 591 | 2.9 | 3.1 |

| | Non-Ar | EB | BZ | Xylene | Heavies | Liquid | Benz/ |
|---|---|---|---|---|---|---|---|
| | Selec. | Selec. | Selec. | Selec. | Selec. | NonAr | Xylene |
| Day | wt % | wt % | wt % | wt % | wt % | wt % | Ratio |
| 1 | 0.9 | 0.5 | 42.4 | 47.6 | 8.6 | 0.8 | 0.89 |
| 2 | 0.9 | 0.5 | 42.4 | 47.7 | 8.5 | 0.6 | 0.89 |
| 3 | 0.9 | 0.6 | 40.5 | 47.6 | 10.4 | 0.5 | 0.85 |
| 6 | 0.8 | 0.7 | 40.2 | 47.7 | 10.6 | 0.5 | 0.84 |
| 7 | 0.8 | 0.5 | 40.6 | 48.5 | 9.6 | 0.4 | 0.84 |
| 8 | 0.8 | 0.5 | 39.7 | 48.9 | 10.1 | 0.4 | 0.81 |
| 9 | 1.3 | 0.5 | 40.6 | 47.9 | 9.6 | 0.4 | 0.85 |
| 10 | 1.3 | 0.5 | 40.3 | 48.2 | 9.7 | 0.4 | 0.84 |
| 13 | 0.7 | 0.5 | 38.5 | 50.2 | 10.1 | 0.3 | 0.77 |
| 14 | 0.6 | 0.5 | 40.2 | 49.1 | 9.6 | 0.4 | 0.82 |
| 15 | 0.9 | 0.6 | 40.0 | 48.6 | 9.9 | 0.4 | 0.82 |
| 16 | 0.9 | 0.6 | 39.7 | 49.0 | 9.9 | 0.4 | 0.81 |
| 17 | 0.9 | 0.6 | 39.1 | 49.3 | 10.2 | 0.4 | 0.79 |
| 20 | 3.2 | 0.6 | 38.1 | 47.8 | 10.3 | 0.4 | 0.80 |
| 21 | 0.7 | 0.6 | 40.4 | 48.3 | 10.0 | 0.4 | 0.84 |
| 22 | 0.7 | 0.5 | 39.4 | 49.3 | 10.0 | 0.4 | 0.80 |
| 23 | 0.8 | 0.5 | 38.8 | 49.6 | 10.2 | 0.3 | 0.78 |
| 24 | 0.8 | 0.5 | 39.2 | 49.5 | 10.0 | 0.4 | 0.79 |
| 27 | 0.7 | 0.5 | 39.8 | 49.2 | 9.7 | 0.4 | 0.81 |
| 29 | 0.9 | 0.6 | 39.1 | 49.2 | 10.3 | 0.3 | 0.79 |
| 30 | 0.5 | 0.5 | 40.2 | 48.7 | 10.0 | 0.4 | 0.83 |
| 31 | 0.8 | 0.6 | 38.1 | 49.9 | 10.6 | 0.3 | 0.76 |
| 34 | 0.9 | 0.5 | 38.5 | 50.0 | 10.1 | 0.3 | 0.77 |
| 35 | 1.4 | 0.5 | 39.4 | 48.8 | 9.9 | 0.4 | 0.81 |

The benzene:xylene ratio for the experimental runs using catalyst without Rhodium is consistently below 0.85.

Additional Rhodium promoted Ni/Mordenite catalyst was prepared using an incipient wetness method as described above wherein a catalyst with 0.01 wt % Rh was prepared and used for Test B and a catalyst with 0.05 wt % Rh was prepared and used for Test C. The following tables provide the results from Test B and C.

TABLE 5
TDP data from Rh—Ni/Mordenite catalyst Test B 0.01 wt % Rh.

| | Toluene | Temp | Pressure | LHSV | H2/toluene |
|---|---|---|---|---|---|
| Day | conversion wt % | ° F. | psig | Hr⁻¹ | molar |
| 1 | 47.5 | 654 | 598 | 3.1 | 1.0 |
| 2 | 44.5 | 654 | 598 | 3.1 | 3.0 |
| 3 | 42.4 | 659 | 598 | 3.2 | 3.0 |
| 4 | 45.1 | 672 | 606 | 3.2 | 3.0 |
| 7 | 45.8 | 672 | 608 | 3.2 | 3.0 |
| 8 | 47.1 | 679 | 596 | 3.2 | 3.0 |
| 9 | 48.5 | 679 | 621 | 3.2 | 3.0 |
| 10 | 49.1 | 679 | 597 | 3.2 | 3.0 |
| 11 | 43.4 | 679 | 597 | 4.6 | 2.1 |
| 14 | 40.8 | 679 | 597 | 4.5 | 2.1 |
| 15 | 39.5 | 679 | 597 | 4.5 | 2.1 |
| 16 | 39.9 | 679 | 600 | 4.6 | 2.1 |
| 17 | 47.6 | 704 | 597 | 4.6 | 2.1 |
| 18 | 44.0 | 704 | 597 | 5.3 | 1.8 |
| 21 | 40.1 | 704 | 597 | 5.1 | 1.9 |
| 23 | 48.2 | 704 | 597 | 3.1 | 3.1 |
| 25 | 46.2 | 704 | 597 | 3.1 | 3.1 |
| 28 | 44.4 | 704 | 597 | 3.6 | 2.6 |
| 29 | 41.4 | 704 | 598 | 4.6 | 2.0 |
| 30 | 41.6 | 704 | 598 | 4.6 | 2.1 |
| 35 | 42.9 | 704 | 598 | 4.6 | 2.1 |
| 36 | 40.9 | 704 | 598 | 3.3 | 2.9 |
| 37 | 40.5 | 704 | 598 | 3.3 | 2.9 |
| 38 | 40.2 | 704 | 598 | 3.3 | 2.9 |
| 39 | 39.7 | 704 | 598 | 3.3 | 2.9 |
| 42 | 40.4 | 704 | 598 | 3.3 | 2.9 |
| 43 | 40.6 | 704 | 598 | 3.3 | 2.9 |
| 44 | 39.9 | 704 | 598 | 3.3 | 2.9 |
| 45 | 39.1 | 704 | 598 | 3.3 | 2.9 |
| 49 | 36.2 | 704 | 597 | 3.3 | 2.9 |
| 52 | 33.8 | 704 | 598 | 3.3 | 2.9 |
| 53 | 33.0 | 704 | 598 | 3.3 | 2.9 |

| | Non-Ar | EB | BZ | Xylene | Heavies | Liquid | Benz/ |
|---|---|---|---|---|---|---|---|
| | Selec. | Selec. | Selec. | Selec. | Selec. | NonAr | Xylene |
| Day | wt % | wt % | wt % | wt % | wt % | wt % | Ratio |
| 1 | 1.0 | 0.5 | 42.3 | 45.2 | 11.0 | 0.7 | 0.94 |
| 2 | 1.8 | 0.4 | 42.8 | 45.8 | 9.2 | 0.6 | 0.93 |
| 3 | 1.5 | 0.4 | 42.6 | 46.0 | 9.5 | 0.6 | 0.93 |
| 4 | 1.4 | 0.5 | 44.6 | 44.6 | 8.9 | 0.6 | 1.00 |
| 7 | 1.1 | 0.4 | 42.6 | 45.4 | 10.5 | 0.5 | 0.94 |
| 8 | 1.1 | 0.5 | 43.0 | 45.2 | 10.2 | 0.5 | 0.95 |
| 9 | 1.4 | 0.5 | 42.8 | 45.5 | 9.7 | 0.5 | 0.94 |
| 10 | 1.4 | 0.4 | 42.0 | 44.2 | 11.9 | 0.5 | 0.95 |
| 11 | 1.1 | 0.3 | 43.6 | 44.9 | 10.1 | 0.5 | 0.97 |
| 14 | 0.9 | 0.3 | 44.0 | 46.9 | 7.9 | 0.4 | 0.94 |
| 15 | 0.9 | 0.2 | 42.6 | 47.5 | 8.6 | 0.4 | 0.90 |
| 16 | 1.0 | 0.2 | 43.5 | 47.5 | 7.8 | 0.4 | 0.92 |
| 17 | 1.1 | 0.4 | 43.1 | 45.4 | 10.1 | 0.4 | 0.95 |
| 18 | 1.2 | 0.3 | 44.0 | 45.6 | 8.8 | 0.4 | 0.97 |
| 21 | 1.0 | 0.3 | 41.8 | 46.7 | 10.1 | 0.2 | 0.90 |
| 23 | 1.3 | 0.5 | 42.8 | 45.0 | 10.5 | 0.4 | 0.95 |
| 25 | 1.3 | 0.4 | 42.2 | 45.2 | 11.0 | 0.3 | 0.93 |
| 28 | 1.1 | 0.4 | 42.6 | 45.8 | 10.1 | 0.3 | 0.93 |
| 29 | 1.0 | 0.3 | 41.5 | 46.5 | 10.7 | 0.3 | 0.89 |
| 30 | 1.1 | 0.3 | 43.8 | 45.2 | 9.7 | 0.4 | 0.97 |
| 35 | 0.6 | 0.3 | 43.3 | 46.2 | 9.6 | 0.3 | 0.94 |
| 36 | 0.8 | 0.3 | 43.6 | 47.0 | 8.2 | 0.3 | 0.93 |
| 37 | 0.9 | 0.3 | 43.0 | 47.6 | 8.2 | 0.3 | 0.90 |
| 38 | 0.9 | 0.3 | 44.2 | 46.9 | 7.7 | 0.3 | 0.94 |
| 39 | 0.9 | 0.3 | 43.8 | 47.1 | 8.0 | 0.3 | 0.93 |
| 42 | 1.0 | 0.3 | 42.9 | 47.7 | 8.2 | 0.3 | 0.90 |
| 43 | 0.7 | 0.2 | 41.9 | 47.0 | 10.1 | 0.3 | 0.89 |
| 44 | 1.0 | 0.2 | 41.9 | 46.7 | 10.2 | 0.3 | 0.90 |
| 45 | 1.0 | 0.2 | 41.9 | 46.6 | 10.2 | 0.3 | 0.90 |
| 49 | 1.3 | 0.2 | 42.2 | 47.2 | 9.2 | 0.4 | 0.89 |
| 52 | 1.5 | 0.2 | 44.2 | 47.8 | 6.4 | 0.4 | 0.92 |
| 53 | 1.3 | 0.2 | 43.5 | 48.8 | 6.3 | 0.4 | 0.89 |

TABLE 6
TDP data from Rh—Ni/Mordenite catalyst Test C 0.05 wt % Rh.

| | Toluene | Temp | Pressure | LHSV | H2/toluene |
|---|---|---|---|---|---|
| Day | conversion wt % | ° F. | psig | Hr⁻¹ | molar |
| 1 | 47.1 | 654 | 598 | 3.1 | 3.0 |
| 2 | 46.8 | 676 | 598 | 3.1 | 3.0 |
| 6 | 43.9 | 662 | 598 | 3.1 | 3.0 |
| 7 | 43.6 | 662 | 599 | 3.1 | 3.0 |
| 9 | 42.1 | 665 | 599 | 3.1 | 3.0 |
| 10 | 45.1 | 666 | 599 | 3.2 | 3.0 |
| 11 | 47.3 | 682 | 599 | 3.2 | 3.0 |
| 13 | 47.6 | 682 | 599 | 3.2 | 3.0 |
| 14 | 44.7 | 682 | 599 | 3.2 | 3.0 |
| 15 | 44.5 | 682 | 599 | 3.2 | 3.0 |
| 16 | 39.0 | 682 | 599 | 3.8 | 2.5 |
| 17 | 37.5 | 682 | 599 | 3.8 | 2.5 |
| 20 | 36.5 | 685 | 599 | 3.8 | 2.5 |
| 21 | 37.7 | 685 | 599 | 3.8 | 2.5 |

| | Non-Ar | EB | BZ | Xylene | Heavies | Liquid | Benz/ |
|---|---|---|---|---|---|---|---|
| | Selec. | Selec. | Selec. | Selec. | Selec. | NonAr | Xylene |
| Day | wt % | wt % | wt % | wt % | wt % | wt % | Ratio |
| 1 | 1.0 | 0.5 | 42.3 | 45.2 | 11.0 | 0.7 | 0.94 |
| 2 | 2.0 | 0.7 | 42.3 | 45.5 | 9.5 | 2.0 | 0.93 |
| 6 | 6.3 | 0.9 | 39.0 | 44.1 | 9.8 | 4.1 | 0.88 |
| 7 | 6.0 | 0.8 | 37.8 | 43.2 | 12.2 | 4.0 | 0.88 |
| 9 | 6.4 | 0.8 | 38.9 | 42.4 | 11.5 | 4.5 | 0.92 |
| 10 | 7.9 | 1.0 | 38.9 | 42.6 | 9.6 | 5.1 | 0.91 |
| 11 | 7.0 | 1.1 | 38.1 | 41.6 | 12.3 | 4.5 | 0.92 |
| 13 | 7.1 | 1.0 | 38.5 | 41.6 | 11.7 | 4.3 | 0.93 |
| 14 | 6.9 | 0.9 | 38.6 | 42.2 | 11.4 | 4.5 | 0.92 |
| 15 | 7.1 | 0.9 | 37.1 | 41.2 | 13.7 | 4.3 | 0.90 |
| 16 | 12.0 | 0.8 | 36.4 | 40.5 | 10.2 | 4.5 | 0.90 |
| 17 | 12.7 | 0.9 | 37.7 | 41.2 | 7.6 | 5.0 | 0.92 |
| 20 | 13.9 | 0.8 | 34.6 | 40.6 | 10.0 | 5.0 | 0.85 |
| 21 | 13.4 | 0.8 | 34.0 | 39.6 | 12.2 | 5.1 | 0.86 |

The benzene:xylene ratio for the TDP experimental runs using Ni/Mordenite catalyst having Rhodium is consistently above 0.85, while the comparative runs using Ni/Mordenite catalyst without Rhodium is consistently below 0.85. A higher benzene:xylene ratio can provide a better benzene selectivity relative to xylene, which can be beneficial in obtaining increased benzene production.

Various terms are used herein, to the extent a term used in not defined herein, it should be given the broadest definition persons in the pertinent art have given that term as reflected in printed publications and issued patents.

The term “activity” refers to the weight of product produced per weight of the catalyst used in a process per hour of reaction at a standard set of conditions (e.g., grams product/gram catalyst/hr).

The term “deactivated catalyst” refers to a catalyst that has lost enough catalyst activity to no longer be efficient in a specified process. Such efficiency is determined by individual process parameters.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

The term “zeolite” refers to a molecular sieve containing a silicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples.

While the foregoing is directed to embodiments of the present invention, other and further embodiments of the invention may be devised without departing from the basic scope thereof and the scope thereof is determined by the claims that follow.

What is claimed is:

1. A catalyst useful in the conversion of hydrocarbons comprising:
a molecular sieve base catalyst promoted with rhodium.

2. The catalyst of claim 1, wherein the molecular sieve catalyst is a zeolite.

3. The catalyst of claim 1, wherein the molecular sieve catalyst is a mordenite zeolite.

4. The catalyst of claim 1, wherein the molecular sieve catalyst is a nickel modified mordenite zeolite.

5. The catalyst of claim 4, wherein the nickel content is between 0.5 wt % and 1.5 wt %.

6. The catalyst of claim 1, wherein the rhodium content is at least 0.005 wt %.

7. The catalyst of claim 1, wherein the catalyst has a silica to alumina molar ratio of from 10:1 to 100:1.

8. The catalyst of claim 1, wherein the catalyst has a silica to alumina molar ratio of from 10:1 to 60:1.

9. The catalyst of claim 1, used in a process for the disproportionation of toluene to benzene and xylene, comprising:
passing a toluene/hydrogen feedstock over the catalyst at reaction conditions sufficient to provide toluene conversion at a rate of about at least 30 percent.

10. The catalyst of claim 9 further comprising:
producing a first product stream comprising benzene and xylene, wherein the benzene: xylene ratio by weight in the first product stream is greater than 0.85.

11. The catalyst of claim 9, wherein the catalyst exhibits extended catalyst life over nickel-mordenite catalyst not promoted with rhodium.

12. The catalyst of claim 9, wherein the reaction temperature ranges from 150° C.-500° C.

13. The catalyst of claim 10, wherein the reaction temperature is adjusted to maintain a toluene conversion level of at least 40 percent.

14. The catalyst of claim 9, wherein the hydrogen:toluene molar ratio is between 0.05:1 to 5:1.

15. The catalyst of claim 9, wherein the hydrogen:toluene molar ratio is between 1:1 to 4:1.

16. The catalyst of claim 9, wherein the reaction pressure range is between 200 psig to 800 psig.

17. The catalyst of claim 9, wherein the toluene conversion reaction can continue with a toluene conversion of at least 30 percent for at least 20 days with no more than 15° C. reactor temperature increase due to catalyst deactivation.

18. The catalyst of claim 9, wherein the toluene conversion reaction can continue with a toluene conversion of at least 40 percent for at least 20 days with no more than 10° C. reaction temperature increase due to catalyst deactivation.

19. The catalyst of claim 9, wherein the catalyst exhibits extended catalyst life by a factor of at least two over nickel-mordenite catalyst not promoted with rhodium.

20. The catalyst of claim 9, wherein the average catalyst deactivation is no more than 0.5° C. per day.

21. The catalyst of claim 1, used in a process for converting a feed of heavy aromatics composed primarily of C₈₊ alkylaromatic compounds to produce products of benzene, toluene and xylene, comprising:
providing a reaction zone containing the nickel-mordenite catalyst promoted with rhodium;
introducing a feed comprising heavy aromatics composed primarily of C₈₊ alkylaromatic compounds at reaction zone conditions; and
removing conversion products from the reaction zone;
wherein the catalyst exhibits extended catalyst life over nickel-mordenite catalyst not promoted with rhodium.

22. The catalyst of claim 21, wherein toluene feed is also introduced into the reaction zone along with the heavy aromatic feed.

23. The catalyst of claim 21, wherein the heavy aromatics make up substantially the entire feed introduced into the reaction zone.

24. The catalyst of claim 21, wherein the heavy aromatics make up at least 75% by total weight of the feed introduced into the reaction zone.

25. The catalyst of claim 21, wherein the reaction zone is operated at a temperature of from about 250° C. to about 500° C., and a pressure of at least 200 psig.

26. The catalyst of claim 21, wherein the average catalyst deactivation is no more than 0.5° C. per day.

27. The catalyst of claim 21, wherein the catalyst exhibits extended catalyst life by a factor of at least two times over nickel-mordenite catalyst not promoted with rhodium.

28. The catalyst of claim 21, further comprising:
introducing a first feed comprising substantially pure toluene feedstock into the reaction zone so that the first feed contacts the catalyst under initial reaction zone conditions selected for the disproportionation of substantially pure toluene to obtain a target toluene conversion between 30% and 55%; and
introducing a second feed comprising heavy aromatics composed primarily of C₈₊ alkylaromatic compounds, allowing conversion of the second feed while the reaction zone is at the reaction zone conditions selected for the disproportionation of the pure toluene.

29. A method for disproportionation of toluene to benzene and xylene, comprising:
passing a toluene and hydrogen feedstock with a hydrogen:toluene molar ratio between 0.05:1 to 4:1 over a nickel-mordenite catalyst promoted with at least 0.005 wt % rhodium at toluene disproportionation conditions to provide toluene conversion at a rate of at least 30 percent;
wherein the catalyst exhibits extended catalyst life over nickel-mordenite catalyst not promoted with rhodium.

30. The method of claim 29, wherein the toluene conversion reaction can continue with a toluene conversion of at least 30 percent for at least 20 days with no more than 15° C. reaction temperature increase due to catalyst deactivation.

31. The method of claim 29, wherein the catalyst exhibits extended catalyst life by a factor of at least two over nickel-mordenite catalyst not promoted with rhodium.

32. The method of claim 29, wherein the average catalyst deactivation is no more than 0.5° C. per day.

33. The method of claim 29, further comprising:
producing a first product stream comprising benzene and xylene, wherein the benzene: xylene ratio by weight in the first product stream is greater than 0.85.

34. A method of converting a feed of heavy aromatics composed primarily of C₈₊ alkylaromatic compounds to produce products of benzene, toluene and xylene, the method comprising:
providing a reaction zone containing a nickel-mordenite catalyst promoted with at least 0.005 wt % rhodium;
introducing a feed comprising heavy aromatics composed primarily of C₈₊ alkylaromatic compounds at reaction zone conditions; and
removing conversion products from the reaction zone;
wherein the catalyst exhibits extended catalyst life over nickel-mordenite catalyst not promoted with rhodium.

35. A method of converting a feed of heavy aromatics composed primarily of C₈₊ alkylaromatic compounds to produce products of benzene, toluene and xylene, the method comprising:
providing a reaction zone containing a nickel-mordenite catalyst promoted with rhodium;
introducing a first feed comprising substantially pure toluene feedstock into the reaction zone so that the first feed contacts the catalyst under initial reaction zone conditions selected for the disproportionation of substantially pure toluene to obtain a target toluene conversion between 30% and 55%;
introducing a second feed comprising heavy aromatics composed primarily of C₈₊ alkylaromatic compounds, allowing conversion of the second feed while the reaction zone is at the reaction zone conditions selected for the disproportionation of the pure toluene;
adjusting reactor conditions to maintain a generally constant reaction severity; and
removing conversion products from the reaction zone;
wherein the catalyst exhibits extended catalyst life over nickel-mordenite catalyst not promoted with rhodium.

36. The method of claim 35, wherein the catalyst exhibits extended catalyst life by a factor of at least two times over nickel-mordenite catalyst not promoted with rhodium.

37. The method of claim 35, wherein the average catalyst deactivation is no more than 0.5° C. per day.
